(19) United States
(12) Patent Application Publication
(10) Pub. No.: US 2004/0101921 A1
(43) Pub. Date: May 27, 2004

(54) Process for label-free measurement of modified substrate

(75) Inventors: Gerald Birk, Warthausen (DE); Steffen Hadamovsky, Biberach/Riss (DE)

Correspondence Address: BOEHRINGER INGELHEIM CORPORATION, 900 RIDGEBURY ROAD, P. O. BOX 368, RIDGEFIELD, CT, 06877, US

(73) Assignee: Boehringer Ingelheim Pharma GmbH & Co. KG, Ingelheim (DE)

(21) Appl. No.: 10/716,125

(22) Filed: Nov. 18, 2003

Related U.S. Application Data
(63) Continuation of application No. 09/823,150, filed on Mar. 30, 2001.

(51) Int. Cl. G01N 33/53; G01N 33/537; G01N 33/543; C12M 1/34

(52) U.S. Cl. 435/7.92; 435/287.2

(57) ABSTRACT

Process for measuring a substance in solutions without first having to selectively label the molecule which is to be measured. The process makes it possible to determine the quantity of the substance, and to measure catalysed reactions which lead to the modification of the substance. The process makes it possible to test whether a substance modulates a catalysed reaction or not. The process can be used in High Throughput Screening.

### RELATED APPLICATION DATA

This application is a continuation of U.S. application Ser. No. 09/823,150 filed Mar. 30, 2001.

### BACKGROUND TO THE INVENTION

The present invention relates to methods of measuring amounts of modified substrate in solutions without the need for prior selective labelling of a molecule which is to be measured. The present invention also relates to the measurement of catalysed reactions which have led to the modification of the substrate. Furthermore, the present invention relates to methods for testing whether a substance has modulated a catalysed reaction or not, particularly methods such as those used in High Throughput Screening (HTS).

The present invention relates in particular to a process for indirectly measuring the quantity of phosphorylated molecules using Reflectometric Interference Spectroscopy (RIfS).

The specific measurement of the quantity of modified substrate in solutions is often used to make predictions as to the quantity of catalysed reactions. Measuring processes are known wherein the substrate is directly labelled during the catalysed reaction, e.g. by the incorporation of radioactive isotopes, dyes, etc., combined with measuring the labelling in a special process, e.g. scintillation measurement, autoradiography, etc. The incorporation of artificial markers of this kind has the disadvantage that in some cases special precautions have to be taken, and they incur particular technical costs. In the case of biological reactions, there is also the disadvantage that non-physiological substrates are produced and the enzymes catalysing the reactions have to react with non-physiological reactants.

A desired method of measurement is one which uses no markers. Marker-free methods of this kind are suitable for a wide spectrum of different biological systems and are therefore also useful for discovering new active substances (Markgren et al. 1999). Methods of label-free detection and characterisation of affinity reactions on surfaces include, for example: changes in frequency of piezoelectric resonators using a quartz microbalance (Ngeh-Ngwainbi et al., 1990; Rickert et al., 1996), changes in frequency of acoustic surface waves using surface wave oscillators (Tom-Moy et al., 1995), changes in the reflectivity of a surface using Brewster angle reflectometry (Stange et al., 1988), measurement of Surface Plasmon Resonance (Striebel et al., 1994).

Reflectometric Interference Spectroscopy (RIfS, Gauglitz et al.) is a method which has already been used to measure DNA-ligand interaction (Piehler et al., 1997), streptavidine-biotin interaction (Piehler et al., 1996b) and for binding immunoprobes to antigens (Piehler et al., 1996a). Brecht et al. 1995 used RIfS in measurements in which antibodies competed for the binding sites on the sensor surface.

It is an aim of the present invention to provide a process for measuring substances which makes it possible to detect label-free modified substrate using an optical measuring system. This process is also suitable for determining the activity of enzymes and for testing substances for their possible modulating effects on enzymatic activity. The process preferably makes it possible to determine kinase activity and substances which modulate this activity.

### DETAILED DESCRIPTION OF THE INVENTION

The present invention permits the label-free measurement of substances which do not have to be given extra labelling in order to measure them, by using physical systems which make molecular interactions measurable. This means systems which detect changes in the phase limits between the probe and transducer. Reflectometric Interference Spectroscopy (RIfS), Plasmon Resonance Spectroscopy (SPR), ellipsometry, the grating coupler and the prism coupler are methods or tools which make such changes measurable. Their physical bases are very different. Whereas SPR measures the shift in the resonance energy of a surface plasmon caused by the change in the refractive index (Liedberg et al., 1983) the prism coupler measures the change in the coupling conditions of an isolated layer waveguide (Cush et al., 1993), the grating coupler measures the shift in the coupling angle of the optical grating (Nellen et al., 1988) and RIfS measures the changes in the interference pattern of a thin layer (Gauglitz et al., 1993). All of them can be used to detect biomolecular interactions, provided that one of the binding partners is immobilised on a transducer surface.

The use of RIfS for measuring modified substrate is posited as an example of the use of physical systems of this kind in the detailed description of the present invention.

The present invention discloses a process for measuring modified substrate which is characterised in that a measuring system comprising a receptor which is capable of binding to a receptor binding site of a substance which is coupled to a sensor surface using a surfactant substance, thereby increasing the layer thickness of the sensor surface, is used in such a way that the receptor is able to bind to a receptor binding site of a modified substrate, the layer thickness of the sensor surface constituting a measurement of the amount of modified substrate.

Accordingly, the present invention relates to a process for measuring modified substrate, characterised in that a sensor surface to which a substance carrying a receptor binding site is coupled is brought into contact, in the presence of a surfactant substance, with a receptor which is capable of binding to this receptor binding site, and with modified substrate to which the receptor can bind, and then the altered layer thickness of the sensor surface is determined.

The “substrate” within the meaning of the present invention is a molecule to which a receptor according to the invention does not bind or binds only weakly. Said substrate can be converted into a “modified substrate” according to the invention by a catalysed reaction.

The term “modified substrate” for the purposes of the present invention denotes a substrate which has been modified by a catalysed reaction and consequently has at least one “receptor binding site” within the meaning of the invention, with the result that a “receptor within the meaning of the invention can bind better to the modified substrate than to the substrate.

A modified substrate within the meaning of the present invention may also be a molecule which has not been changed by a catalysed reaction if it was already capable of binding to a receptor via a receptor binding site.

The term “receptor” within the meaning of the present invention denotes a molecule which can bind to a “receptor binding site” of a substance. Such receptors include, for example, protein complexes, proteins, peptides, nucleic acid sequences and other organic and inorganic molecules. Antibodies and fragments thereof which can bind to a receptor binding site are preferred. Particularly preferred is an antibody, or a fragment thereof, which can bind to phosphotyrosine.

The term “receptor binding site” of a substance within the meaning of the present invention denotes a partial structure of a molecule to which a receptor can bind. Receptor binding sites with equal binding specificity are found within the meaning of the present invention on modified substrate and on correspondingly structured sensor surface. In the case of equal binding specificity, a univalent receptor within the meaning of the present invention binds either to the receptor binding site of the modified substrate or to the receptor binding site of the substance which is bound to the sensor surface. Competitive binding takes place.

Also within the meaning of the invention are receptor binding sites with different binding specificity to modified substrate and to correspondingly structured sensor surface. In this case, a bivalent receptor can bind to both different receptor binding sites and thereby bind the modified substrate to the sensor surface via the receptor. This double binding may also occur within the meaning of the invention in the case of receptor binding sites with equal binding specificity using a di- or multivalent receptor.

A process according to the present invention comprises the use of a so-called “surfactant” which is capable of reducing or preventing non-specific binding of a molecule (e.g. a receptor, a substrate, a modified substrate, an enzyme or other substances which may be used in the processes according to the invention), particularly non-specific binding to a sensor surface and/or- receptor binding site used when carrying out the present invention.

The preferred surfactant is Brij 35 (CAS number 9002-92-0), as this substance shows absolutely no non-specific binding to the sensor surface and is therefore particularly suitable as a surfactant for use in determining the layer thickness of the sensor. Brij 35 Solution (Messrs. Sigma) is particularly preferred.

The preferred substance is preferably used in a suitable concentration which the skilled person can easily determine by series of titrations. A concentration of the surfactant Brij 35 of 0.5-0.002% (v/v) is particularly preferred.

The concentrations specified in the Examples are most particularly preferred.

A preferred process is characterised in that the modified substrate is a protein.

Also preferred is a process which is characterised in that the layer thickness of the sensor surface is determined by Reflectometric Interference Spectroscopy (RIfS).

Also preferred is a process the measuring system of which comprises a receptor which can bind to a receptor binding site of a substance which is bound to the sensor surface, and at the same time can bind to an identical or different receptor binding site on the modified substrate.

More preferable is a process the measuring system of which comprises a receptor which can only bind to one receptor binding site, while the receptor binding site which is bound to the sensor surface is identical to the receptor binding site on the modified substrate.

Even more preferable is a process which uses

a) an antibody as receptor

a) phosphotyrosine as the receptor binding site

a) a modified substrate, preferably one selected from among (i) phosphorylated Poly (Glu,Tyr) 4:1(CAS number 97105-00-5), (ii) phosphorylated oligopeptide Ac-Ile-Tyr-Gly-Phe-NH₂, particularly preferably phosphorylated oligopeptide Ac-Ile-Tyr-Gly-Phe-NH₂ “M-2165” (Messrs. Bachem), and (iii) phosphorylated protein-tyrosine-kinase substrate, particularly preferably phosphorylated protein-tyrosine-kinase substrate Raytide™ EL (Messrs. Calbiochem), and

a) a sensor to which a substance with a receptor binding site is coupled, selected from among aminodextrane (AMD) sensor and-diamino PEG 2000 sensor.

A protein-tyrosine-kinase substrate within the meaning of the present invention is a chemical compound which may be phosphorylated by protein-tyrosine-kinases.

A sensor within the meaning of the present invention is a conventional glass transducer. The “aminodextrane (AMD) sensor” or “diamino PEG 2000 sensor” known from the prior art (e.g. Schütz A., 2000; a) Piehler J., et al, 1996; Universität Tübingen, Inst. f Physikalische and Theoretische Chemie, Germany) is preferred.

Another embodiment is a process wherein the modified substrate is the result of at least one enzymatic reaction which is to be detected, which has preceded the measurement of the layer thickness. A process in which the modified substrate is the result of precisely one enzymatic reaction Which is to be detected is particularly preferred. More preferable is a process wherein the enzymatic reaction is a reaction of phosphorylation.

Even more preferable is a process which uses

a) an antibody as receptor

b) phosphotyrosine as the receptor binding site

c) a phosphorylatable substrate, preferably one selected from among (i) Poly (Glu,Tyr) 4:1(CAS number 97105-00-5), (ii) oligopeptide Ac-Ile-Tyr-Gly-Phe-NH₂, particularly preferably oligopeptide Ac-Ile-Tyr-Gly-Phe-NH₂ “M-2165” (Messrs. Bachem), and (iii) protein-tyrosine-kinase substrate, particularly preferably protein-tyrosine-kinase substrate Raytide™ EL (Messrs.

Calbiochem), and

d) a kinase preferably selected from among (i) p60c-src-kinase, particularly preferably p60c-scr-kinase (isolated from human cells, Messrs. Calbiochem), and (ii) EGF-receptor-kinase (EGF-RK), particularly preferably EGF-receptor-kinase (Messrs. Sigma), and

e) a sensor to which a substance with a receptor binding site is coupled, selected from among AMD sensor and diamino PEG 2000 sensor.

Another embodiment of the present invention is a process wherein the enzymatic activity of a protein under investigation is determined by measuring the modified substrate. Thus, using the test kit according to the invention, it is possible to investigate whether a protein has a kinase activity, for example.

According to another aspect, the present invention relates to a test kit for measuring enzymatic activity, preferably kinase activity, of a protein under investigation, which consists of

a) a receptor which is capable of binding to a receptor binding site, preferably an antibody which is particularly preferably capable of binding to phosphotyrosine,

b) a sensor to which a substance with a receptor binding site can be coupled, preferably an AMD sensor or a diamino PEG 2000 sensor, and

c) a substrate to which the receptor can bind after its conversion into a modified substrate with a receptor binding site. The substrate is preferably selected from among (i) Poly (Glu,Tyr) 4:1(CAS number 97105-00-5), (ii) oligopeptide Ac-Ile-Tyr-Gly-Phe-NH₂, particularly preferably oligopeptide Ac-Ile-Tyr-Gly-Phe-NH₂ “M-2165” (Messrs. Bachem), and (iii) protein-tyrosine-kinase substrate, particularly preferably protein-tyrosine-kinase substrate Raytide™ EL (Messrs. Calbiochem), for use in a process according to the invention by which an enzymatic activity of a protein can be determined. The positive control according-to the invention may be a kinase, preferably EGF-RK or p60c-src-kinase.

According to another aspect the invention relates to the use of a test kit according to the invention to determine whether a substance activates or inhibits an enzymatic reaction.

The present invention also relates to the use of a process according to the invention for determining whether a substance activates or inhibits an enzymatic reaction, characterised in that in a process according to the invention wherein an enzymatic reaction which precedes the measurement of the layer thickness is carried out in the presence of a substance which is to be tested.

In a preferred process according to the invention one or more substances which are to be tested are present in order to determine whether one of these substances modulates an enzymatic reaction.

Particularly preferred is a process according to the invention which determines whether the modulating activity is an activating one.

Also preferred is a process according to the invention which determines whether the modulating activity is an inhibiting one.

It is particularly preferred to use processes according to the invention in High Throughput Screening (HTS), characterised in that a plurality of measurements are made in parallel, and the sensor surfaces can be used several times.

The use of Reflectometric Interference Spectroscopy (RIfS) according to the invention allows label-free measurement of the interactions between reactants. The measurement of the change in layer thickness on the surface of the sensor is sufficiently quick, reproducible and reliable. Repeated regeneration allows the sensor to be used over and over again, dramatically reducing the working material required and the waste, with a consequent significant reduction in costs.

The method of measurement according to the invention is based on the interaction between an immobilised substance having a receptor binding site and a free receptor. This arrangement has advantages over a process in which the receptor is immobilised. Thus, even small substances can be measured using the process according to the invention, as the attachment of any larger receptor thereto by binding causes an increase in layer thickness which is easily measured.

The present invention is particularly suitable for measuring kinase activity or for determining the modulation of kinases.

Kinases are enzymes which regulate metabolic processes in many ways. In order to understand such correlations and discover possible therapeutically active substances for treating illnesses which can be helped by the modulation of kinases, it is helpful to use systems which will detect a reaction of phosphorylation and its possible modulation by suitable substances.

The present invention provides highly sensitive processes which are particularly suitable for this purpose.

The phosphorylation of the polyamino acid Poly-(Glu,Tyr) 4:1 by EGF-RK demonstrates the possibilities for using RIfS technology in screening. The use of the process according to the invention also makes it possible to detect the phosphorylation of even tiny substrates without any difficulty. In addition, it opens up the possibility of using substrates or fragments of natural substrates which are syhthesised or modified in any way which might be envisaged. The skilled man will be familiar, from the prior art, with alternatives to the kinases, substrates, antibodies and excipients described here which constitute an equivalent means of solving the problem solved here.

All the embodiments disclosed in the Examples are preferred embodiments of the present invention.

### EXAMPLES

The following Examples illustrate the present invention by way of example.

### Example 1

Measurement of Modified Substrate, in this Case Phosphorylated Substrate, by Way of Example

1. The Starting Materials for the Phosphorylation Assay:

1.1. EGF-Receptor Kinase (EGF-RK) Buffer:

EGF-Receptor-Kinase (EGF-RK) Buffer:

The buffer contains, in the final concentration:

25 mM of Tris HCl (molecular weight (MW) MW: 121.1; Messrs. Sigma)

10 mM MgCl₂; (MW: 203.3; Messrs. Merck)

First, the above substances are dissolved in distilled water. The pH is adjusted to 7.60 with dilute hydrochloric acid. Then 100 μM of Na₃VO₄; (MW: 183.9 Messrs. Sigma) already dissolved in a small amount of water are added, followed by 2 mM of 2-mercaptoethanol (MW: 78.13, Messrs. Sigma). Then the pH of the buffer is adjusted to 7.50 with dilute hydrochloric acid. The volume is then made up to the specified end volume. The buffer is stored in the refrigerator and can be kept for at least 1 week.

The surfactant Brij 35 is added to the buffer as described in 1.7.

1.2. p60c-src-Kinase-Buffer:

The p60c-src-kinase buffer contains in the final concentration 250 mM of Hepes (MW: 238.3; Messrs. Sigma) and 150 mM of MgCl2 (MW: 203.3; Messrs. Merck) with a pH of 7.40.

The buffer may be prepared, for example, by first dissolving the abovementioned substances in distilled water, adjusting the pH with dilute sodium hydroxide solution to a pH of 7.40 and then topping it up to the end volume.

This buffer is used for the incubation mixture. For rinsing and washing the sensors 100 ml of-the buffer are diluted with 400 ml of distilled water, producing the final concentrations which are also found in the incubation mixture for the phosphorylation assay, namely 50 mM of Hepes and 30 mM of magnesium chloride.

1.3. ATP Solution:

Adenosine 5′ triphosphate disodium salt (MW: 551.1, Messrs. Sigma), ATP, is dissolved in a concentration of 1 mM in distilled water.

The solution is made up into 200 μl aliquots and stored at −20° C. until ready for use.

1.4. Substrate for the Enzymatic Reaction, in this Case the Reaction of Phosphorylation:

1.4.1. Poly-(Glu,Tyr) 4:1

Poly-(Glu,Tyr) 4:1 (MW: 39100, Messrs. Sigma) is dissolved in a concentration of 20 μg/10 μl in EGF-RK-buffer pH=7.50. The solution is made up into 200 μl aliquots and stored at −20° C. until ready for use.

1.4.2. Raytide™ EL

Raytide™ EL (MW: 2476 Dalton; Messrs. Calbiochem) is dissolved in distilled water in a concentration of 1 μg/10 μl. The solution is made up into 100 μl aliquots and stored at −20° C. until ready for use.

1.4.3. M-2165

5 mg of the oligonucleotide Ac-Ile-Tyr-Gly-Glu-Phe-NH₂, [“M-2165” made by Messrs Bachem (MW: 668.8) being used here], are combined with 2 ml of distilled water.

After the addition of 20 μl of a 10% ammonia solution, the substances dissolves completely. It is then topped up with 2.980 ml of distilled water to give the final volume of 5.0 ml.

The solution is made up into 100 μl aliquots and stored at −20° C. until ready for use.

1.5. Enzyme Producing an Enzymatic Reaction, in this Case: Kinases for Reaction of Phosphorylation:

1.5.1. Epidermal Growth Factor Receptor Kinase (EGF-RK)

The kinase, EGF-RK isolated from human A 431 carcinoma cells (Messrs. Sigma) being used here, is dissolved in EGF-RK buffer in a concentration of 2 units/10 μl, pH=7.50.

The solution is made up into 100 μl aliquots and stored at −70° C. until ready for use.

1.5.2. p60c-src-Kinase

The kinase, p60c-src-kinase isolated from human cells (Messrs. Calbiochem) being used here, is dissolved in a concentration of 2 units/10 μl, pH=7.40, in Hepes buffer with 50 mM of Hepes and 30 mM of magnesium chloride.

The solution is made up into 40 μl aliquots and stored at −20° C. until ready for use.

1.6. Receptor Which is Capable of Binding a Receptor Binding Site, in this Case an Antibody:

The antibody, the monoclonal murine antibody IgG1 from Ascites: Anti-PhosphoTyrosine (Clone number: PT-66, Messrs. Sigma) being used here, is dissolved in a concentration of 1 μg/10 μl in 10 mM of Hepes buffer, pH=7.00. The solution is made up into 200 μl aliquots and stored at −70° C. until ready for use.

1.7. Surfactant for Reducing Non-Specific Binding to the Sensor Surface:

The surfactant Brij 35 (CAS 9002-92-0), [Brij 35 Solution (Messrs. Sigma) being used in the Examples], is used in a concentration of 0.015% of Brij 35 (v/v) unless stated otherwise.

1.8. Preparation of a Sensor to Which a Substance with a Receptor Binding Site is coupled

1.8.1. The Immobilisation of Fmoc-Phospho-Tyrosine (CAS 147762-53-6) On Sensor Surfaces:

A conventional aminodextrane (AMD) sensor or a diamino PEG 2000 sensor is used (e.g. Schütz A., 2000; a) Piehler J., et al., 1996; Universität Tübingen, Inst. f. Physikalische and Theoretische Chemie, Germany.)

Phospho-tyrosine cannot be immobilised directly, as there is a danger of crosslinking and imprecisely defined immobilisation on the sensor surface on account of the reaction with TBTU (O-benzotriazol-1-yl-N,N,N′,N′,-tetramethyl-uronium-tetrafluoroborate; MW: 321.1; Messrs. Sigma) and DIPEA (N,N-diisopropylethyl-amine; MW: 129.2; Messrs. Sigma). For this reason the immobilisation of the phospho-tyrosine has to be done by a slightly circuitous method. The phospho-tyrosine is provided with the Fmoc protecting group which can easily be cleaved again after the immobilisation has been effected. The Fmoc-phospho-tyrosine is commercially obtainable e.g. from Messrs. Bachem; Order No. B-2470.

I) Immobilisation of Fmoc-Phosphotyrosine with TBTU and DIPEA:

1.: solution 1: 20 mg of TBTU are dissolved in 200 μl of dimethylformamide (DMF).

2.: solution 2: 15 mg of Fmoc-phosphotyrosine are dissolved in 200 μl of solution 1.

3.: solution 3: 20 μl of DIPEA are pipetted into 200 μl of solution 2.

20 μl aliquots of the final solution are immediately added drop by drop to the sensor surface which is about 1 cm² in size, and incubated for 8 hours under DMF chamber saturation. Then the sensor is rinsed with DMF and dried in the air.

II) Cleaving of the Fmoc Protecting Group with Piperidine

A 20% (v/v) solution in DMF is prepared from the piperidine (Messrs. Fluka). 20 μl aliquots of this solution are added drop by drop to the sensor surface and incubated for 15 minutes. Then the sensor is rinsed with DMF and dried in the air.

Leaving the piperidine to act for any longer has a detrimental effect on the sensor.

It is not absolutely necessary to cleave the Fmoc protecting group as the receptor mentioned under 1.6. recognises the receptor binding site, the phospho-tyrosine, even with the Fmoc protecting group.

The sensors are stored in the refrigerator until ready for use and may be used for several months.

1.8.2. Regeneration of the Sensor Surface After a Measurement

The sensor is regenerated with a solution of pepsin in hydrochloric acid. The pepsin solution is made fresh every day. 40 mg of pepsin (Messrs. Sigma) are dissolved in 20 ml distilled water. The pH of the solution is adjusted to 1.90 with dilute hydrochloric acid.

50 μl of this regeneration solution are pipetted into the measuring cell on the sensor surface. The regeneration time is 120 seconds in this case, depending on the strength of the interaction of the receptor with the receptor binding site, as can easily be determined by anyone skilled in the art. Then the measuring cell with the sensor is thoroughly washed twice with a buffer in the same dilution as is used in the subsequent measurement (e.g. p60c-src-kinase buffer diluted 1:5; cf 1.2.) to remove the regeneration solution completely.

2. Carrying Out the Measurement or Carrying Out the Phosphorylation and Subsequent Measurement:

Before the start of the experiment, 0.015% (v/v) of Brij 35 is added to the buffer solutions used, unless otherwise stated. The EGF-RK buffer is used unless otherwise stated.

The desired aliquots of substrate, ATP and the antibody, and optionally the enzyme, are thawed at ambient temperature.

50 μl of EGF-RK buffer with or without Brij 35 (as specified in each case) are placed in a 500 μl microreaction vessel with a lid. The quantity depends mainly on the volumes which are still to be pipetted, the buffer being intended to make the volume of the phosphorylation assay up to the total of 100 μl .

Then 10 μl of 10% (v/v) DMSO solution are added, or if a substance is to be tested for possible enzyme-modulating properties, 10 μl of a test substance, i.e. a possible inhibitor or activator, dissolved in 10% (v/v) of DMSO, are pipetted in.

If an enzymatic reaction is to take place, the enzyme is added at this stage. In the case of EGF-RK, for example, between 1-4 units in 20 μl of EGF-RK buffer are added.

In the case of p60c-src-kinase, for example, between 2-6 units in μl of p60c-src-kinase buffer are added.

Then the substrate is added. The amounts of the substrates Poly-(Glu,Tyr) 4:1, M-2165 and Raytide™ EL used are specified in each case.

The reaction of phosphorylation is started here by the addition of 10 μl of the 1 mM ATP solution.

The incubation time is 30 minutes at +30° C., for example.

The incubation takes place in an Eppendorf thermostatically controlled heating block.

The samples are mixed repeatedly using the agitator.

After the end of the incubation period, 20 μl of antibody solution (1.6.) are pipetted in, for example. The receptor, in this case the antibody, is used in excess. The amount which corresponds to an excess can very easily be determined by anyone skilled in the art by means of experiments on binding to a sensor surface to which a substance with a receptor binding site is coupled, with increasing amounts of receptor. In the case of reactions of phosphorylation, increasing amounts of receptor are used with a constant amount of substrate and kinase.

The incubation time with the antibody may be, for example, 30 minutes at ambient temperature.

The samples are mixed repeatedly using the agitator.

After the end of the incubation period 50 μl of incubation mixture are taken and pipetted into the prepared measuring cell with the sensor to whose surface is coupled a substance with a receptor binding site.

The layer thickness of the sensor is measured, for example, by Reflectometric Interference Spectroscopy (RIfS) using a SPEKOL 1100 simultaneous spectral photometer made by Messrs Zeiss Jena, modified according to Schmitt et al. 1997.

By means of the change in layer thickness as a result of the binding of the receptor, in this case the antibody, to the receptor binding site of the sensor surface, suitable experiments are carried out to determine

(a) the quantity of a modified substrate, e.g. phosphorylated Poly-(Glu,Try)4:1, phosphorylated Raytide™ EL, phosphorylated M-2165,

(b) the modification of a substrate, e.g. phosphorylation of e.g. Poly-(Glu,Try)4:1, Raytide™ EL, M-2165,

(c) an enzymatic activity of an enzyme, e.g. kinases, e.g. EGF-RK, p60c-src-kinase and

(d) the modulating effect of a test substance on an enzymatic activity.

The receptor (in this case the antibody) is used in excess, so that even if there is a total enzymatic reaction (in this case phosphorylation) of the substrate at least a slight binding signal will still be obtained by the binding of the receptor (antibody) to the sensor surface.

3. Changing the Layer Thickness of the Sensor:

3.1. Layer Thickness Formation at Different Concentrations of the Substrate Poly-(Glu,Tyr) 4:1

Different amounts of Poly-(Glu,Tyr) 4:1 are added to a diamino-PEG 2000 sensor to which a substance with a receptor binding site, in this case phosphotyrosine, is bound and the layer thickness is measured as described in 2.

The buffer used is EGF-RK buffer.

The following amounts of Poly-(Glu,Tyr) 4:1 are used:

TABLE 1
Non-specific binding of Poly-(Glu, Tyr) 4:1 (PGT)
to the phospho-tyrosine-diamino-PEG 2000
sensor surface.

| | Optical layer thickness in nm |
|---|---|
| 0.5 μg PGT | 0.064 |
| 1.0 μg PGT | 0.026 |
| 2.0 μg PGT | 0.000 |
| 5.0 μg PGT | 0.052 |
| 10 μg PGT | 0.065 |
| 20 μg PGT | 0.054 |
| 50 μg PGT | 0.113 |
| 100 μg PGT | 0.084 |

The substrate Poly-(Glu,Tyr) 4:1 binds only slightly to the phospho-tyrosine-diamino-PEG 2000-sensor surface even at very high concentrations.

3.2. Layer Thickness Formation by the Substrate Raytide™ EL in the Presence of Brij 35.

The experiments carried out within the scope of the invention show that Raytide™ EL binds non-specifically to sensors according to the invention. It is necessary to a find a substance which reduces this binding.

Raytide EL (1.0 μg/50 μl) is added together with increasing concentrations of Brij 35 to an AMD sensor to which a substance with a receptor binding site, in this case phosphotyrosine, is bound and the layer thickness is measured as described under 2.

The buffer used is p60c-src-kinase buffer, pH=7.40, with the final concentrations of 50 mM of Hepes and 30 mM of magnesium chloride in the incubation mixture.

The following concentrations of Brij 35 (% v/v) are used:

TABLE 2
Increase in layer thickness as a result of Raytide ™ EL
in the presence of Brij 35.

| | Optical layer thickness in nm |
|---|---|
| Raytide ™ EL without Brij 35 | 0.169 |
| with 0.001% Brij 35 | 0.067 |
| with 0.002% Brij 35 | 0.083 |
| with 0.01% Brij 35 | 0.000 |
| with 0.02% Brij 35 | 0.000 |
| with 0.1% Brij 35 | 0.000 |
| with 0.2% Brij 35 | 0.000 |

Even small concentrations of Brij 35 reduce the non-specific binding of the substrate Raytide™ EL. At rather higher concentrations the non-specific binding of the substrate is prevented entirely.

3.3. Layer Thickness Formation by the Receptor, Phospho-Tyrosine Antibody in the Presence of Brij 35.

It has astonishingly been found, within the scope of the invention, that Brij 35 prevents unwanted non-specific binding (cf. 3.2.). An investigation is carried out to see whether Brij 35 prevents the desired interaction of the receptor with the receptor binding site, in this case the binding of the phosphotyrosine antibody to the sensor surface to which phosphotyrosine is coupled.

Antibody (1.0 μg/50 μl) is added together with increasing amounts of Brij 35 to an AMD sensor to which a substance with a receptor binding site, in this case phosphotyrosine, is bound and the layer thickness is measured as described in 2.

The buffer used is p60c-src-kinase buffer, pH=7.40, with the final concentrations of 50 mM of Hepes and 30 mM of magnesium chloride in the incubation mixture.

The concentrations of Brij 35 (% v/v) shown below are used:

TABLE 3
Increase in layer thickness as a result of the phosphotyrosine
antibody in the presence of Brij 35.

| | Optical layer thickness in nm |
|---|---|
| antibody without Brij 35 | 2.838 |
| with 0.001% Brij 35 | 2.540 |
| with 0.002% Brij 35 | 2.629 |
| with 0.01% Brij 35 | 2.931 |
| with 0.02% Brij 35 | 3,000 |
| with 0.1% Brij 35 | 2.789 |
| with 0.2% Brij 35 | 2.660 |

The surfactant Brij 35 has no significant influence on the desired binding of the phospho-tyrosine antibody to the phospho-tyrosine-AMD sensor surface.

3.4. EGF-RK

3.4.1. Layer Thickness Formation at Different Concentrations of EGF-R-Kinase.

Different amounts of EGF-RK are added to a diamino-PEG 2000 sensor to which a substance with a receptor binding site, in this case phosphotyrosine, is bound and the layer thickness is measured as described in 2. The buffer used is EGF-RK buffer. As a control, buffer solution without the enzyme EGF-RK-is measured.

TABLE 4
Epidermal Growth Factor Receptor kinase (EGF-RK)
binding to the phospho-tyrosine-diamino-PEG 2000
sensor surface

| | Optical layer thickness in nm |
|---|---|
| control value | 0.000 |
| 1 Unit/50 μl | 0.829 |
| 2 Units/50 μl | 1.744 |
| 3 Units/50 μl | 2.134 |
| 4 Units/50 μl | 1.358 |
| 5 Units/50 μl | 1.144 |

The EGF-RK binds to a considerable degree to the phospho-tyrosine-diamino-PEG 2000 sensor surface. It is striking that, as the concentration rises, the non-specific binding first increases, then decreases.

Such high non-specific binding militates against the detection of phosphorylation.

3.4.2. Layer Thickness Formation by EGF-RK in the Presence of Brij 35.

Experiments carried out within the scope of the invention showed that EGF-RK binds non-specifically to the surfaces of the sensors according to the invention (3.4.1.). It is essential to reduce this non-specific binding.

Two different amounts of EGF-RK (1 Unit/50 μl and 2 Units/50 μl) together with 0.01% v/v of Brij 35 are added to a diamino-PEG 2000 sensor to which a substance with a receptor binding site, in this case phosphotyrosine, is bound and the layer thickness is measured as described in 2. The buffer used is EGF-RK buffer. As a control, buffer solution without EGF-RK is measured.

TABLE 5
Epidermal Growth Factor receptor kinase (EGF-RK)
binding to the phospho-tyrosine-diamino-PEG 2000
sensor surface in the presence of Brij 35 (% v/v)

| | Optical layer thickness in nm |
|---|---|
| 1 Unit/50 μl | 0.627 |
| 1 Unit/50 μl + 0.01% Brij 35 | 0.000 |
| 2 Units/50 μl | 1.941 |
| 2 Units/50 μl + 0.01% Brij 35 | 0.000 |

The non-specific binding of EGF-RK can be totally prevented by Brij 35.

3.5. Layer Thickness Formation by p60c-src-Kinase in the Presence of Brij 35.

The layer thickness formation of p60c-src-kinase (1 Unit/50 μl) in the presence of Brij 35 is investigated. This is done using Hepes buffer pH=7.40 with 50 mM of Hepes and 30 mM of magnesium chloride. p60c-src-kinase is added together with different amounts of Brij 35 to an AMD sensor, to which a substance with a receptor binding site, in this case phosphotyrosine, is bound and the layer thickness is measured as described in 2. Increasing concentrations of Brij 35 (% v/v) are added to the buffer.

TABLE 6
Increase in layer thickness as a result of p60c-src-kinase
in the presence of Brij 35

| | Optical layer thickness in nm |
|---|---|
| kinase without Brij 35 | 2.414 |
| with 0.001% Brij 35 | 1.818 |
| with 0.002% Brij 35 | 1.622 |
| with 0.01% Brij 35 | 0.939 |
| with 0.02% Brij 35 | 0.775 |
| with 0.1% Brij 35 | 0.445 |
| with 0.2% Brij 35 | 0.599 |

The surfactant Brij 35 is capable of significantly reducing the non-specific binding of p60c-src-kinase;

4. Phosphorylation Assay

4.1. Enzyme EGF-RK

4.1.1 Layer Thickness Formation by Epidermal Growth Factor (EGF) in the Presence of Brij 35.

It has repeatedly been stated in the literature that EGF is advantageous or necessary as an activator for phosphorylation with EGF-RK.

An investigation is carried out to see whether EGF binds non-specifically to the receptor binding site of the sensor according to the invention in the presence of Brij 35.

To do this, varying amounts of EGF (0.5 μg/50 μl and 1.0 μg/50μ) are added together with 0.01% v/v of Brij 35 to a diamino-PEG, 2000 sensor to which a substance with a receptor binding site, in this case phosphotyrosine, is bound and the layer thickness is measured as described in 2. The buffer used is EGF-RK buffer.

TABLE 7
Epidermal Growth Factor (EGF) binding to the
phospho-tyrosine-diamino-PEG 2000 sensor surface.

| | Optical layer thickness in nm |
|---|---|
| EGF 0.5 Units | 0.814 |
| EGF 0.5 Units + Brij 35 | 0.320 |
| EGF 1.0 Units | 0.833 |
| EGF 1.0 Units + Brij 35 | 0.484 |

The non-specific binding of EGF can only be reduced by half with the concentration of Brij 35 used. An investigation is therefore carried out to see whether the presence of EGF is absolutely necessary in the reaction of phosphorylation.

4.1.2. EGF Phosphorylation of the Substrate Poly-(Glu,Tyr) 4:1 by EGF-RK at a Reduced Kinase Concentration and Presence of Epidermal Growth Factor

In experiments carried out within the scope of the invention it was found that EGF binds non-specifically to the surfaces of the sensors according to the invention in the presence of Brij 35. To check whether the activator EGF has an influence on EGF-RK, an experiment is carried out with a reduced kinase concentration and EGF.

Poly-(Glu,Tyr) 4:1 used in a concentration of 10 μg/50 μl, EGF-RK in a concentration of 0.1 Unit/50 μl and EGF in a concentration of 0.5 μg/50 μl are added to a diamino-PEG 2000 sensor to which a substance with a receptor binding site, in this case phosphotyrosine, is bound, and the layer thickness is measured as described in 2. EGF-RK buffer with Brij 35 is used as the buffer. The 1^{st} mixture without ATP contains all the abovementioned ingredients needed for the phosphorylation with the exception of ATP. This mixture is thus termed the “control”. The 2^{nd} mixture contains all the abovementioned ingredients as well as ATP, so that phosphorylation can take place.

First, the antibody, then the kinase, the substrate and EGF are measured on their own in order to be able to evaluate any non-specific binding.

TABLE 8
Binding of the receptor (antibody) before and after
phosphorylation of Poly-(Glu, Tyr) 4:1 by EGF-RK
in the presence of EGF

| | Optical layer thickness in nm |
|---|---|
| Mixture without ATP | 2.589 |
| Complete mixture | 2.037 |
| antibody on its own | 3.044 |
| EGF-R on its own | 0 |
| substrate on its own | 0 |
| EGF on its own | 0.355 |

The phosphorylation is still detectable. Since only a little substrate was phosphorylated, more antibody binds to the phospho-tyrosine-diamino-PEG 2000 sensor surface. The use of Epidermal Growth Factor did not confer any recognisable advantage. EGF binds non-specifically in spite of the presence of Brij 35. Since the activation described in the literature is only about 15%, the non-specific binding would neutralise this advantage again. Since the EGF-R-kinase is present in an already activated form, the use of EGF appears to be pointless and unnecessary.

4.1.3. Phosphorylation of the Substrate Poly-(Glu,Tyr) 4:1 by EGF-RK in the Absence of EGF

Poly-(Glu,Tyr) 4:1 in a concentration of 10 μg/50 μl, EGF-RK used in a concentration of 2 Units/50 μl are added to a diamino-PEG 2000 sensor to which a substance with a receptor binding site, in this case phosphotyrosine, is bound, and the layer thickness is measured as described in 2. EGF-RK buffer with Brij 35 is used as the buffer.

The 1^{st} mixture without ATP contains all the abovementioned ingredients needed for. the phosphorylation with the exception of ATP. This mixture is thus termed the “control”. The 2^{nd} mixture contains all the abovementioned ingredients as well as ATP, so that phosphorylation can take place.

First, the antibody, then the kinase and the substrate are measured on their own in order to be able to evaluate any non-specific binding.

TABLE 9
Binding of the receptor (antibody) before and after
phosphorylation of Poly-(Glu, Tyr) 4:1 by EGF-RK

| | Optical layer thickness in nm |
|---|---|
| Mixture without ATP | 2.651 |
| Complete mixture | 0.225 |
| antibody on its own | 3.158 |
| EGF-RK on its own | 0.183 |
| substrate on its own | 0 |

The phosphorylation can be detected very clearly. The non-specific binding of the kinase is very slight. No non-specific binding of the substrate can be detected. There is no need for EGF.

Further Experiments:

The substrate concentration is 10 μg/50 μl, the kinase concentration is 1 Unit/50 μl. The buffer used is EGF-RK buffer with Brij 35. The 1^{st} mixture and the 8^{th} mixture without ATP contain all the abovementioned ingredients needed for the phosphorylation with the exception of ATP. All the other mixtures contain all the abovementioned ingredients including ATP, so that the phosphorylation can take place.

TABLE 10
Phosphorylation of Poly-(Glu, Tyr) 4:1 by EGF-RK.

| | Optical layer thickness in nm |
|---|---|
| Mixture without ATP (1) | 2.268 |
| Complete mixture (2) | 0.146 |
| Complete mixture (3) | 0.205 |
| Complete mixture (4) | 0.182 |
| Complete mixture (5) | 0.185 |
| Complete mixture (6) | 0.199 |
| Complete mixture (7) | 0.225 |
| Mixture without ATP (8) | 2.057 |

TABLE 11
Mean values and standard deviation of the data in Table 10

| | Mixture without ATP | Complete mixture |
|---|---|---|
| | optical layer thickness | optical layer thickness |
| | in nm | in nm |
| | | 0.146 |
| | | 0.205 |
| | 2.268 | 0.182 |
| | 2.057 | 0.185 |
| | | 0.199 |
| | | 0.225 |
| mean value | 2.162 | 0.190 |
| standard | 0.149 | 0.027 |
| deviation | | |

The phosphorylation can be shown up very clearly and reproducibly.

4.1.4. Phosphorylations with Alternative Substrate

4.1.4.1. Phosphorylation of the Substrate Raytide™ EL by EGF-R-K

To check how suitable other substrates are for phosphorylation by EGF-R-kinase, the Poly-(Glu,Tyr) 4:1 used hitherto is replaced by the substrate Raytide™ EL.

Raytide™ EL is used in a concentration of 2 μg/50 μl together with EGF-RK (0.5 Units/50 μl) on a diamino-PEG 2000 sensor to which a substance with a receptor binding site, in this case phosphotyrosine, is bound, and the layer thickness is measured as described in 2. The buffer used is EGF-RK buffer with Brij 35. The 1^{st} mixture without ATP contains all the abovementioned ingredients needed for the phosphorylation with the exception of ATP. This mixture is thus termed the “control”. The 2^{nd} mixture contains all the abovementioned ingredients including ATP, so that phosphorylation can take place.

First, the antibody, then the kinase and the substrate are measured on their own in order to be able to evaluate any non-specific binding.

TABLE 12
Binding of the receptor (antibody) before and after
phosphorylation of Raytide ™ EL by EGF-RK

| | Optical layer thickness in nm |
|---|---|
| Mixture without ATP | 3.113 |
| Complete mixture | 1.833 |
| antibody on its own | 3.015 |
| EGF-R on its own | 0.043 |
| substrate on its own | 0.127 |

The phosphorylation with the alternative substrate Raytide™ EL is clearly detectable.

4.2. Phosphorylation of an Alternative Substrate; M-2165 by an

Alternative Kinase; p60c-src-Kinase on an Alternative Sensor—Measurement with the Aminodextrane (AMD) Sensor.

Use of alternative substrates, kinase and sensors in a process according to the invention. M-2165 is used in a concentration of 10 μg/50 μl together with p60c-src-kinase (4 Units/50 μl) on an AMD sensor to which a substance with a receptor binding site, in this case phosphotyrosine, is bound, and the layer thickness is measured as described in 2. The buffer used is Hepes buffer pH=7.40 with 50 mM of Hepes and 30 mM of magnesium chloride. The buffer contains 0.5% v/v mercaptoethanol, 0.05% v/v Brij 35 and 10 μg/50 μl albumin.

The 1^{st} mixture without ATP contains all the abovementioned ingredients needed for the phosphorylation with the exception of ATP. This mixture is thus termed the “control”. The 2^{nd} mixture contains all the abovementioned ingredients including ATP, so that phosphorylation can take place.

First, the antibody, then the kinase and the substrate are measured on their own in order to be able to evaluate any non-specific binding.

TABLE 13
Binding of the receptor (antibody) before and after
phosphorylation of M-2165 by p60c-src-kinase to the
phospho-tyrosine-aminodextrane sensor surface.

| | Optical layer thickness in nm |
|---|---|
| Mixture without ATP | 3.813 |
| Complete mixture | 1.104 |
| antibody on its own | 2.500 |
| kinase on its own | 0.719 |
| substrate on its own | 0.230 |

The phosphorylation is very clearly detectable. The non-specific binding of the kinase and of the substrate is slight.

4.2. Phosphorylation of the Substrate M-2165 by p60c-src-Kinase—Measurement on the Diamino-PEG 2000 Sensor.

M-2165 is used in a concentration of 10 μg/50 μl together with p60c-src-kinase (2 Units/50 μl) on a diamino-PEG 2000 sensor to which a substance with a receptor binding site, in this case phosphotyrosine, is bound, and the layer thickness is measured as described in 2. The buffer used is Hepes buffer pH=7.40 with 50 mM of Hepes and 30 mM of magnesium chloride. The buffer contains 0.5% v/v of mercaptoethanol, 0.05% v/v of Brij 35 and 10 μg/50 μl of albumin.

The 1^{st} mixture without ATP contains all the abovementioned ingredients needed for the phosphorylation with the exception of ATP. This mixture is thus termed the “control”. The 2^{nd} mixture contains all the abovementioned ingredients including ATP, so that phosphorylation can take place.

First, the antibody, then the kinase and the substrate are measured on their own in order to be able to evaluate any non-specific binding.

TABLE 13 a
Binding of the antibody before and after phosphorylation
of M-2165 to the phospho-tyrosine-diamino-PEG 2000
sensor surface

| | Optical layer thickness in nm |
|---|---|
| Mixture without ATP | 3.202 |
| Complete mixture | 1.064 |
| antibody on its own | 2.99 |
| kinase on its own | 0.501 |
| substrate on its own | 0.285 |

The phosphorylation is very clearly detectable. The non-specific binding of the kinase is still somewhat less. The experiment is easily reproducible.

### Example 2

4.3. Measuring the Inhibition of Phosphorylation Mediated by EGF-RK

4.3.1. Inhibition of Phosphorylation by the Inhibitor Genistein (CAS 446-72-0)

Poly-(Glu,Tyr) 4:1 in a concentration of 10 μg/50 μl, EGF-RK in a concentration of 1 Unit/50 μl together with various concentration of genistein are added to a diamino-PEG 2000 sensor to which a substance with a receptor binding site, in this case phosphotyrosine, is bound and the layer thickness is measured as described in 2. The buffer used is EGF-RK buffer with Brij 35.

The genistein is added after the pipetting of the kinase, then the substrate is added. The reaction of phosphorylation is started by the addition of ATP.

At the start of the experiment a control and a complete phosphorylation without the addition of inhibitor are carried out.

TABLE 14
Inhibition of the phosphorylation of Poly-(Glu, Tyr) 4:1 by
EGF-RK with genistein.

| | Optical layer thickness in nm |
|---|---|
| Mixture without ATP | 2.400 |
| Complete mixture | 0.446 |
| 10-8M genistein | 0.758 |
| 10-7M genistein | 0.994 |
| 10-6M genistein | 1.465 |
| 10-5M genistein | 1.782 |
| 10-4M genistein | 2.232 |

Inhibition by genistein is clearly detectable. The IC₅₀ is estimated to be in the region of 10⁻⁶M, which also agrees with data in the literature.

4.3.2. Inhibition of Phosphorylation by the Inhibitor Tyrphostin 47 (CAS 118409-60-2)

Poly-(Glu,Tyr) 4:1 in a concentration of 10 μg/50 μl, EGF-RK in a concentration of 1 Unit/50 μl together with various concentrations of Tyrphostin 47 are added to a diamino-PEG 2000 sensor to which a substance with a receptor binding site, in this case phosphotyrosine, is bound, and the layer thickness is measured as described in 2. The buffer used is EGF-RK buffer with Brij 35.

The Tyrphostin 47 is added after the pipetting of the kinase, then the substrate is added. The reaction of phosphorylation is started by the addition of ATP.

At the start of the experiment a control and a complete phosphorylation without the addition of inhibitor are carried out.

TABLE 15
Inhibition of the phosphorylation of
Poly-(Glu, Tyr) 4:1 by EGF-RK with Tyrphostin 47.

| | Optical layer thickness in nm |
|---|---|
| Mixture without ATP | 2.304 |
| Complete mixture | 0.613 |
| 10-8M Tyrphostin | 0.578 |
| 10-7M Tyrphostin | 0.630 |
| 10-6M Tyrphostin | 0.847 |
| 10-5M Tyrphostin | 1.685 |
| 10-4M Tyrphostin | 2.344 |

Inhibition by Tyrphostin 47 is clearly apparent. The IC₅₀ is in the range from 10⁻⁶M to 10⁻⁵M , which agrees with the data from the literature.

4.3.3. Inhibition of Phosphorylation by the Inhibitor Erbstatin Analogue (CAS 63177-57-1)

Poly-(Glu,Tyr) 4:1 in a concentration of 10 μg/50 μl, EGF-RK in a concentration of 1 Unit/50 μl together with various concentrations of erbstatin analogue are added to a diamino-PEG 2000 sensor to which a substance with a receptor binding site, in this case phosphotyrosine, is bound, and the layer thickness is measured as described in 2. The buffer used is EGF-RK buffer with Brij 35.

The erbstatin is added after the pipetting of the kinase, then the substrate is added. The reaction of phosphorylation is started by the addition of ATP.

At the start of the experiment a control and a complete phosphorylation without the addition of inhibitor are carried out.

TABLE 16
Inhibition of the phosphorylation of
Poly-(Glu, Tyr) 4:1 by EGF-RK with
erbstatin analogue

| | Optical layer thickness in nm |
|---|---|
| Mixture without ATP | 2.226 |
| Complete mixture | 0.852 |
| 10-8M erbstatin analogue | 0.526 |
| 10-7M erbstatin analogue | 0.489 |
| 10-6M erbstatin analogue | 0.442 |
| 10-5M erbstatin analogue | 0.702 |
| 10-4M erbstatin analogue | 0.728 |
| 10-3M erbstatin analogue | 1.170 |

The inhibition by erbstatin analogue is incomplete even at higher concentrations. Erbstatin is also described as a weak inhibitor in the literature.

4.3.4. Inhibition of Phosphorylation by the Inhibitor Staurosporin (CAS 62996-74-1)

Poly-(Glu,Tyr) 4:1 in a concentration of 10 μg/50 μl, EGF-RK in a concentration of 1 Unit/50 μl together with various concentrations of staurosporin are added to a diamino-PEG 2000 sensor to which a substance with a receptor binding site, in this case phosphotyrosine, is bound, and the layer thickness is measured as described in 2. The buffer used is EGF-RK buffer with Brij 35.

The staurosporin is added after the pipetting of the kinase, then the substrate is added. The reaction of phosphorylation is started by the addition of ATP.

At the start of the experiment a control and a complete phosphorylation without the addition of inhibitor are carried out.

TABLE 17
Inhibition of the phosphorylation of
Poly-(Glu, Tyr) 4:1 by EGF-RK with
staurosporin

| | Optical layer thickness in nm |
|---|---|
| Mixture without ATP | 2.993 |
| Complete mixture | 0.233 |
| 10-10M staurosporin | 0.190 |
| 10-9M staurosporin | 0.196 |
| 10-8M staurosporin | 0.336 |
| 10-7M staurosporin | 1.030 |
| 10-6M staurosporin | 1.748 |

Inhibition by staurosporin is clearly detectable. The IC₅₀ is estimated to be in the region of 10⁻⁶M, which also agrees with the data in the literature.

### LITERATURE

1) Brecht, Andreas; Piehler, Jacob; Lang, Gerd; Gauglitz, Günter; A direct optical immunosensor for atrazine detection; Analytica Chimica Acta, Vol.311. p.289-299, 1995.

2) Brecht, Andreas; Burckardt, Ralph; Rickert, Jan; Stemmler, Ivo; Schuetz, Armin; Fischer, Stefan; Friedrich, Thomas; Gauglitz, Guenter; Goepel, Wolfgang; Transducer-Based Approaches for Parallel Binding Assays in HTS; Journal of Biomolecular Screening, Vol.1. No.4, p.191-201. 1996.

3) Cush R., Cronin J. M., Stewart W. J., Maule C H., Molly J., Goddard N. J. The resonant mirror: a novel optical biosensor for direct sensing of biomolecular interactions. Part I: principle of operation and associated instrumentation, Biosens. Bioelectron, Vol. 8, 347-353. 1993.

4) Gauglitz G., Brecht A., Kraus G., Nahm W. Chemical and biochemical sensors based on interferometry at thin (multi-) layers, Sens. Actuators, B Vol.11. 21-27, 1993.

5) Hermanson,Greg T.; Mallia, A. Krishna; Smith, Paul K.; Immobilized Affinity Ligand Techniques; Academic Press; 1997

6) Hermanson, Greg T.; Bioconjugate Techniques; Academic Press; 2000

7) Liedberg B., Nylander C., Lundstrom I., Surface plasmon resonance for gas detection and biosensing, Sens. Actuators, Vol. 4, 299-304, 1983.

8) Markgren P. O., Hamalainen M., Danielson U. H., Screening of compounds interacting with HIV-1 proteinase using optical biosensor technology, Anal. Biochem., Vol. 265, 340-350. 1999.

9) Nellen P H., Teifenthaler K., Lukosz W. Integrated optical input grating couplers as biochemical sensors, Sens. Actuators Vol.15, 285-295, 1988.

10)Ngeh-Ngwainbi J., Suleiman A. A., Guibault G. G. , Piezoelectric crystal biosensor, Biosens. Bioelectron. Vol. 5,13-26, 1990.

11)Piehler, Jacob; Modifizierung von Oberflächen fur die thermodynamische und kinetische Charakterisierung biomolekularer Erkennung mit optischen Transducern Dissertation Universität Tübingen; Shaker Verlag, Aachen; 1997.

12) a) Piehler, Jacob; Brecht, Andreas; Geckeler, Kurt E.; Gauglitz, Günter; Surface modification for direct immunoprobes; Biosensors & Bioelectronics Vol.11. No.6/7, pp.579-590, 1996.

13) b) Piehler, Jacob; Brecht, Andreas; Gauglitz, Günter; Affinity Detection of Low Molecular Weight Analytes; Anal.Chem. Vol.68, p.139-143. 1996.

14)Piehler, Jacob; Brecht, Andreas; Gauglitz, Günter; Zerlin, Marion; Maul, Corinna; Thiericke, Ralf; Grabley, Susanne; Label-Free Monitoring of DNA-Ligand Interactions; Analytical Biochemistry, Vol.249, p 94-102. 1997.

15)Rickert. J., Weiss T., Kraas W. Jung G., Goepel W., A new affinity biosensor: Self-assembled thiols as selective monolayer coating of quartz crystal microbalances, Biosens. Bioelectron. Vol., 11. 591-598, 1996.

16) Schmitt H. -M., Brecht A., Piehler J., Gauglitz G. An integrated system for optical biomolecular interaction analysis, Biosen. Bioelectron. Vol.12. 219-233. 1997.

17) Schutz, Armin; Einsatz der Reflectometrischen Interferenzspektroskopie (RIfS) zur markierungsfreien Affinitatsdetektion für das Hochdurchsatzscreening; Dissertation Universität Tübingen; 2000.

18) Stange U., Hutchins M. G., Groome N., Tarassenko L, Quantitative analysis of immunological reactions on silicon surfaces by multiple-angle Brewster angle reflectometry, Biomaterials Vol. 9, 58-61. 1988.

19) Striebel Ch., Brecht A., Gauglitz, G., Characterization of biomembranes by spectral ellipsometry, surface plasmon resonance and interferometry with regard to biosensor application, Biosens. Bioelectron. Vol. 9, 139-146, 1994.

20)Tom-Moy M., Baer R. L., Spira-Solomon D., Doherty T. P., Atrazin measurement using surface transverse wave devices, Anal. Chem. Vol. 67, 1510-1514, 1995.

21)Weetall, Howard H.; Immobilized Enzymes, Antigens, Antibodies, and Peptides; Marcel Dekker Inc.; New York 1975.

What is claimed is:

1. A process for measuring a modified substrate, comprising:
contacting a sensor surface selected from aminodextrane (AMD) sensor and diamino PEG 2000 sensor, to which a substance carrying a phosphotyrosine receptor binding site is coupled, in the presence of a surfactant substance,
with a receptor which is an antibody capable of binding to the receptor binding site and with a modified substrate selected from phosphorylated Poly (Glu,Tyr) 4:1, phosphorylated Ac-Ile-Tyr-Gly-Glu-Phe-NH₂, and phosphorylated Lys-Lys-Lys-Gly-Pro-Trp-Leu-Glu-Glu-Glu-Glu-Glu-Ala-Tyr-Gly-Trp-Leu-Asp-Phe, to which the receptor can bind;
determining by Reflectometric Interference Spectroscopy (RIfS) an altered layer thickness of the sensor surface;
wherein the receptor can bind to only one receptor binding site, and wherein the receptor binding site of the substance which is bound to the sensor surface is identical to the receptor binding site on the modified substrate.

2. The process according to claim 1 wherein the modified substrate is a result of one enzymatic phosphorylation reaction which is to be measured which has preceded the measurement of the layer thickness;
wherein the enzyme is a kinase selected from p60c-src-kinase and EGF-receptor-kinase (EGF-RK).

3. The process according to claim 1 wherein the modified substrate is phosphorylated Poly (Glu,Tyr) 4:1.

4. The process according to claim 1 wherein the modified substrate is phosphorylated Ac-Ile-Tyr-Gly-Glu-Phe-NH₂.

5. The process according to claim 1 wherein the modified substrate is phosphorylated Lys-Lys-Lys-Gly-Pro-Trp-Leu-Glu-Glu-Glu-Glu-Glu-Ala- Tyr-Gly-Trp-Leu-Asp-Phe.
